(19) United States
(12) Patent Application Publication
(10) Pub. No.: US 2019/0076051 A1
(43) Pub. Date: Mar. 14, 2019

(54) SYSTEM AND METHOD FOR GUIDANCE OF A CATHETER TIP

(71) Applicant: MEDICAL COMPONENTS, INC., Harleysville, PA (US)

(72) Inventors: Michael Joyce, Philadelphia, PA (US); Andres B. Cruz, JR., Red Hill, PA (US)

(21) Appl. No.: 15/699,258

(22) Filed: Sep. 8, 2017

(51) Int. Cl. A61B 5/06; A61B 5/0452; A61B 5/044; A61B 5/00

(52) U.S. Cl. CPC A61B 5/065; A61B 5/6852; A61B 5/044; A61B 5/0452

(57) ABSTRACT

A system and method for guiding the placement of a peripherally inserted central catheter in a patient is provided. The system includes a peripherally inserted central catheter; at least one sensor configured to be coupled to said catheter and structured to sense and transmit an electrical signal associated with a P wave; a data base including known data corresponding to standard and non-standard P waves including peaks, deflections and transition points; and a processing device in operable communication with said at least one sensor and said data base, said processing device configured to receive said electrical signal and identify P waveform attributes of said signal, cross-reference said waveform attributes and said electrical signal with known data in said base data, and output indicia representative of said P waveform attributes and said signal based on said cross-reference.

### FIELD OF THE INVENTION

The invention relates generally to a system and method for guidance of a peripherally inserted central catheter within the vasculature of a patient. More particularly, the system includes ECG signal-based catheter tip guidance, in the form of a graphical display, to enable a user to identify a P-wave and guide the catheter tip to an ideal anatomical position with respect to a node of the patient's heart from which the ECG signals originate.

### BACKGROUND OF THE INVENTION

Electrocardiograms may be used for monitoring peripherally inserted central catheter tip locations near the heart. Atrial and ventricular depolarization and repolarization are represented on the ECG as a series of waves: the P wave followed by the QRS complex and the T wave. The first deflection is the P wave associated with right and left atrial depolarization. In practice, a user who is positioning a peripherally inserted central catheter will monitor catheter placement on an electrocardiogram as the ECG or EKG tracks the deflection of the P wave as the catheter is advanced from the upper ⅓ of the superior vena cava down to through the lower ⅓ of the superior vena cava, and into the right atrium. Pulses from the Sinoatrial Node are detected by the PICC as it enters the superior vena cava. The impulse grows stronger as the tip advances down the vein, signified by an enlarged p-wave visible on the ECG. Passing the node causes a negative deflection in the wave, indicating to the user that the tip is positioned beyond the cava-atrial junction and into the right atrium, and should be retracted. The ideal tip position for a PICC line is between the distal superior vena cava and within one centimeter before the right atrium. The ideal tip position is referred to as the transition point between the peak of the P wave and the negative deflection.

However, ECG devices rely on subjective P wave interpretations by medical personnel that may easily be misinterpreted. The foregoing method of identifying the ideal anatomical position for the catheter tip is not suitable for all patient groups. It is well documented that obese, the young and patients with AF will not obtain a clear P wave rise. Also the presence of a pacemaker will mean that the P wave size is not affected by the PICC tip position. In addition, not all P waves exhibit the same patterns making misinterpretation of the transition point likely. Further, an abnormal P wave may preclude use of P wave interpretation entirely. Misinterpretation of the transition point may lead to malpositioning of the catheter tip with the associated risks and complications. In addition, the transition point or landing zone for the catheter tip may vary up to 5 cm.

Therefore, what is needed is a reliable system and method that allows the user to easily confirm the peak, deflection and transition points of a P wave.

### BRIEF SUMMARY OF THE INVENTION

The foregoing problems associate with interpreting P waves are addressed by the system and method in accordance with the invention.

In one aspect, the system and method includes a sensor that is positioned on the tip of a peripherally inserted central catheter (PICC) or in a lumen thereof.

In another aspect of the invention, the system includes a graphical display that depicts information relating to the catheter during insertion into the body. An icon or other indicia provides a graphical representation of the three-dimensional position of the catheter.

In another aspect of the invention a system for guiding the placement of a peripherally inserted central catheter is disclosed. The system broadly includes a control processor for converting a P wave signal into graphical information representative of the three-dimensional location of the catheter within the patient's body; a display that depicts the graphical information; and indicia depicted on the display that indicates a three-dimensional position of the catheter.

In another aspect the graphical representation guides the placement of a peripherally inserted central catheter making it relatively easy and intuitive to determine the ideal catheter tip location during a medical procedure without having to interpret an ECG wave form transition.

In another aspect, the simple gauge allows for ready usage in any electronic display used for catheter tip placement.

In another aspect of the invention the system includes at least one sensor capable of sensing the electrical signal associated with the P wave. The sensor is configured for attachment on the tip of the peripherally inserted central catheter or in a lumen thereof and configured to provide a waveform signal to a processing device. The waveform signal may comprise a P wave including the initial deflection, the maximum height or peak, a negative deflection and a transition point. The transition point is the point between the negative deflection and the peak and indicates correct positioning of the PICC. The system may also include a database including known data corresponding to standard and non-standard P waves including peaks, deflections and transition points thereof. A processing device is in operable communication with the sensor and the database and is configured to receive the at least one waveform signal and calculate waveform attributes of the waveform including the initial deflection of a P wave, the maximum peak, the secondary deflection and the transition point. The processing device further compares the waveform attributes with the database of known data and output a graphical representation of the maximum peak, the secondary deflection and the transition point. The system in accordance with the invention also includes a display device configured to display the output and may comprise a computer screen, a flat-screen display, a projector, a printing device and/or an audible device. The display device may also have user input device configured to communicate with the display device and the processing device.

In another aspect of the invention a method for guiding the placement of a peripherally inserted central catheter in a patient is provided. The method includes providing a peripherally inserted central catheter having a tip and a lumen therewithin; coupling at least one sensor to said catheter for sensing and transmitting an electrical signal associated with a P wave; providing a data base including known data corresponding to standard and non-standard P waves including peaks, deflections and transition points; providing a processing device for communicating with said at least one sensor and said data base, said processing device for receiving said electrical signal from said at least one sensor and identifying P waveform attributes of said signal, cross-referencing said waveform attributes and said electrical signal with known data in said base data, and outputting indicia representative of said P wave and P waveform attributes based on said cross-reference

### BRIEF DESCRIPTION OF THE DRAWINGS

For a better understanding of the invention, and to show how the same may be carried into effect, reference will now be made, by way of example, to the accompanying drawings, in which:

FIG. 1 is a view illustrating a typical wave pattern associated with atrial and ventricular depolarization and repolarization.

FIGS. 2A and 2B illustrates various positions A-E within the heart and the associated P waves.

FIGS. 3A-3C illustrate several abnormalities associated with P waves showing deflections that may be mistaken for the P wave deflection from which the transition point can be derived.

FIG. 4 is a schematic diagram of the system of the present invention illustrating the components thereof.

FIGS. 5A-5C depicts bar graphs generated by the system and method in accordance with the invention for P wave interpretation.

FIG. 6 is a flow chart illustrating the method for P wave interpretation in accordance with the invention.

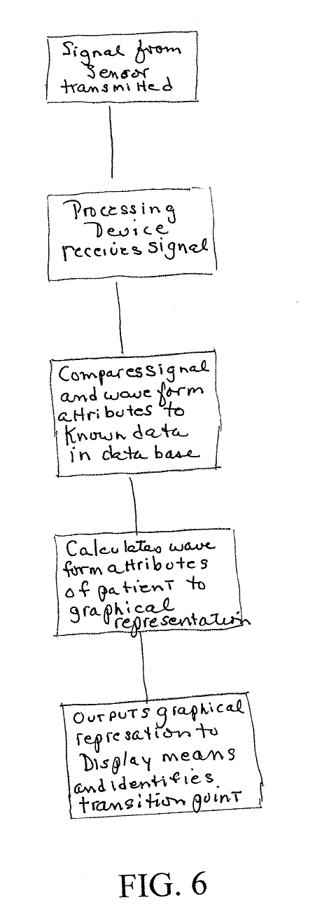

### DETAILED DESCRIPTION OF THE INVENTION

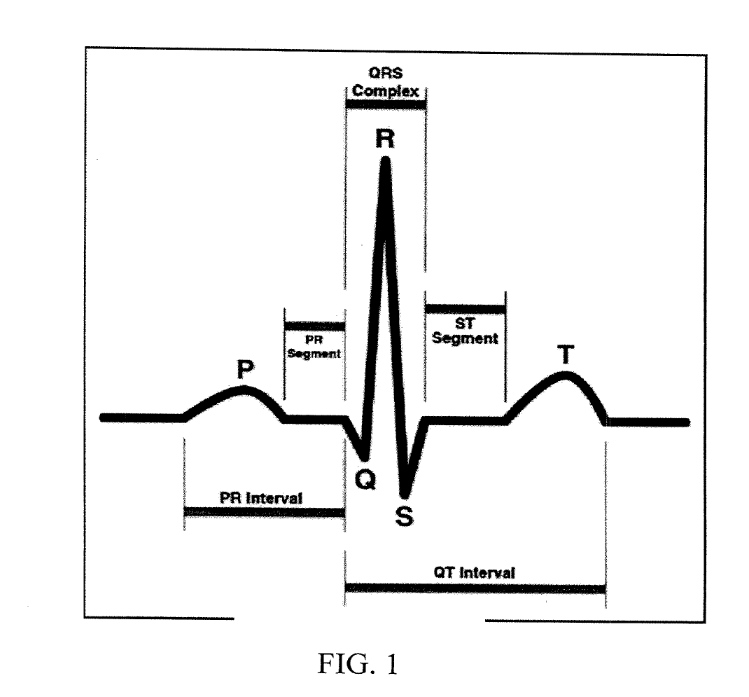

Embodiments of the present invention are generally directed to a catheter placement system configured for accurately placing a catheter within the vasculature of a patient. Referring to FIG. 1 the various patterns of the P wave are depicted.

ECG signal-based catheter tip guidance is included in the system in accordance with the invention that enables guidance of the catheter tip to a desired position with respect to a node of the patient's heart from which the ECG signals originate.

The catheter placement system in accordance with the invention facilitates catheter placement within the patient's vasculature with a relatively high level of accuracy, i.e., placement of the distal tip of the catheter in a predetermined and desired position. Moreover, because of the ECG-based guidance of the catheter tip, correct tip placement may be confirmed without the need for a confirmatory X-ray. This, in turn, reduces the patient's exposure to potentially harmful x-rays, the cost and time involved in transporting the patient to and from the x-ray department, costly and inconvenient catheter repositioning procedures, etc.

Referring now to FIG. 1 there is shown an ECG waveform of atrial and ventricular depolarization and repolarization. Normal ECG tracings consist of waveform components that indicate electrical events during one heart beat. The waveforms are P, Q, R, S T and U. The P wave is the first deflection and is normally a positive (upward) waveform that indicates atrial depolarization. The P-wave looks like a small bump upwards from the baseline. The amplitude is normally 0.05 to 0.25 mV (0.5 to 2.5 small boxes). Normal duration is 0.06-0.11 seconds (1.5 to 2.75 small boxes). The shape of a P-wave is usually smooth and rounded. However, depending on the individual a P wave may be inverted and may have a shape that varies from the standard P wave. The first deflection is the P wave associated with right and left atrial depolarization. A user will typically monitor catheter placement on the electrocardiogram as the ECG or EKG tracks the deflection of the P wave as the catheter is advanced from the upper ⅓ of the superior vena cava down to through the lower ⅓ of the superior vena cava, and into the right atrium. The change in the P wave correlates to the position of the catheter tip. When the catheter tip reaches the lower ⅓ of the superior vena cava/right atrial junction, the P wave reaches its maximum height. The P wave is reduced (a deflection) as the catheter tip is pulled back from the entrance to the right atrium. Thus, the negative deflection of the P wave, which is not always viewable on an ECG tracing, indicates to a user that the catheter tip is terminated at the junction of the right atrium and the superior vena cava. This point corresponds to the transition point between the maximum P wave and the negative deflection and it the point for correct placement of the PICC.

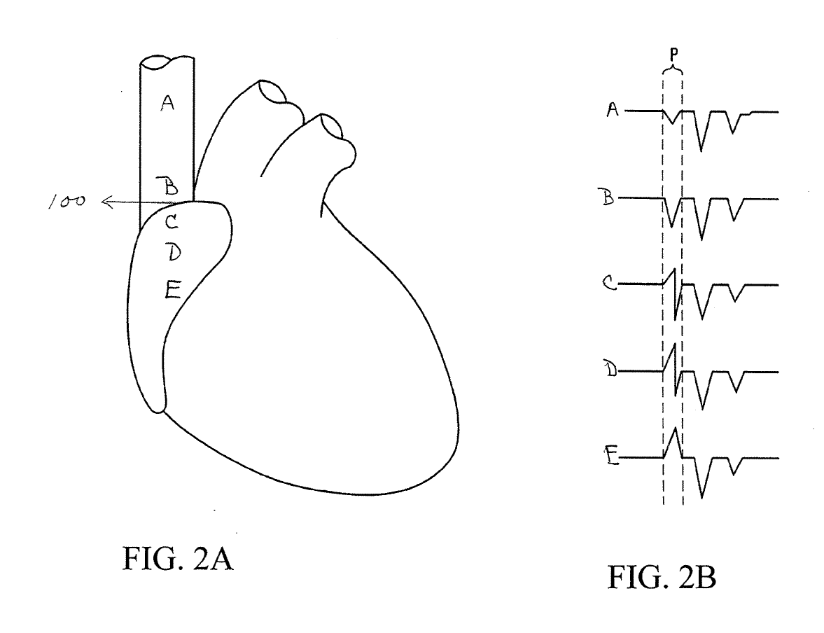

Referring now to FIGS. 2A and 2B various catheter tip positions A-E within the heart and the associated P waves are depicted. The P wave portions of the ECG traces of FIG. 2B are labeled with the letter “P” and occur between the vertical dashed lines. Each of the exemplary traces is numbered to correspond to positions A through E of FIG. 2A Therefore, the ECG trace A was produced when the catheter tip was located in the superior vena cava. The trace B was produced when the catheter tip was in position B. The trace C was produced when the catheter tip was adjacent to the sino-atrial node in the right atrium.

As the electrode attached to PICC tip is advanced further into the right atrium, the polarity of the P wave “P” changes from predominantly negative near the top of the right atrium (position C) to isoelectric (i.e., half has a positive polarity and half has a negative polarity) near the middle of the right atrium (position D) to almost entirely positive at the bottom of the right atrium (position E). Correct anatomical positioning of the catheter tip is at transition point 100 between the distal superior vena cava and within one centimeter before the right atrium.

To ensure correct placement of the catheter tip the method and system in accordance with the invention will display the peak, deflection and transition points of a P wave on display means.

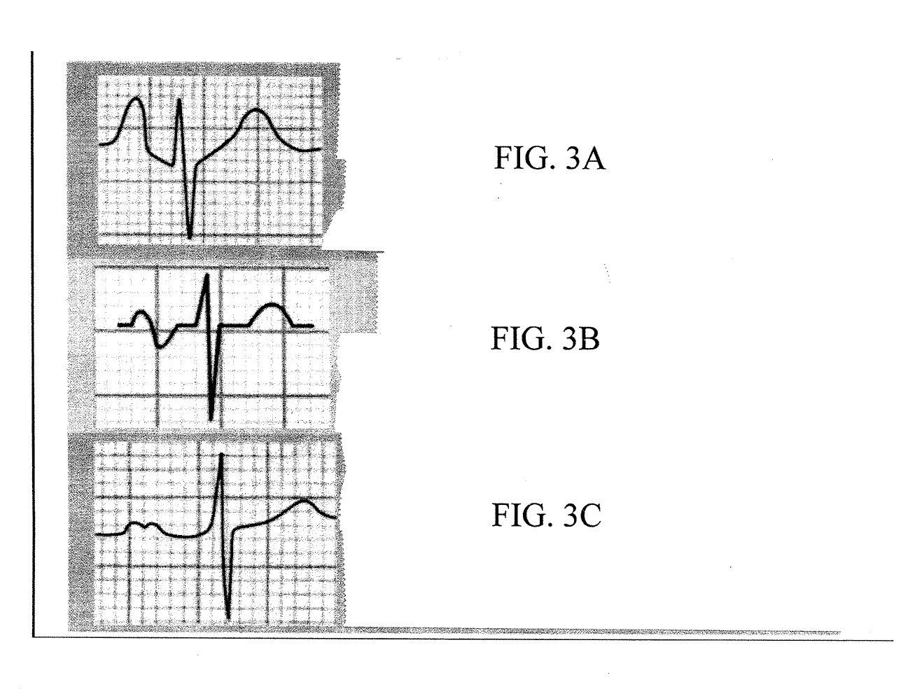

Referring to FIGS. 3A-C a variety of abnormal P waves are depicted that may preclude a user from correctly identifying the transition point without the benefit of the method and system in accordance with the invention. FIG. 3A is a P pulmonale or tall peaked P wave generally due to an enlarged right atrium associated with congenital heart disease, tricuspid valve disease and/or pulmonary hypertension. FIG. 3B is a biphasic P wave with the terminal negative deflection more than 40 ms wide indicating left atrial enlargement. FIG. 3C is a P mitrale or wide P wave, often bifid, and due to mitral stenosis.

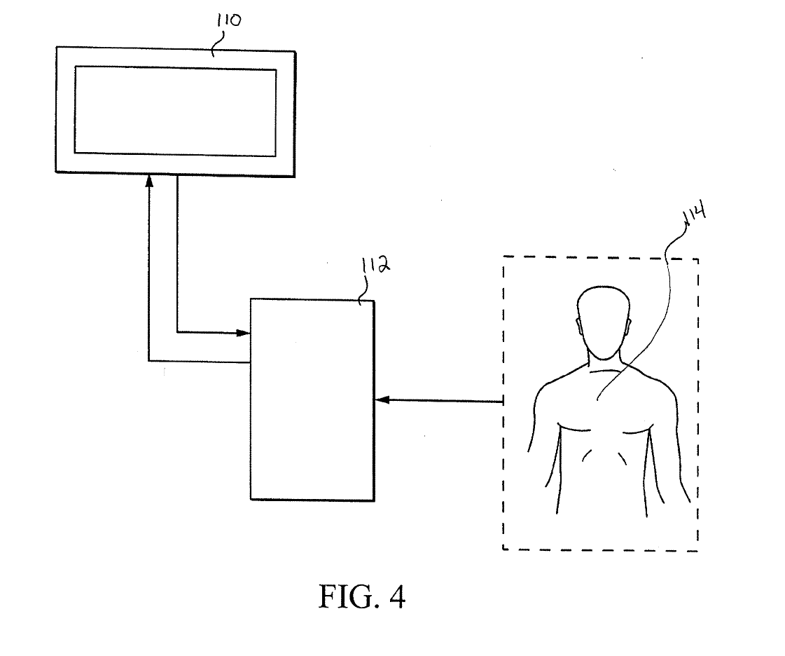

Referring now to FIG. 4, one embodiment of the system in accordance with the present invention includes a display device 110 used to display graphical representation of a P wave including the peak, the deflection point and a processing device 112. Processing device 112 receives inputs from a sensor 114 that coupled to a tip or a lumen of a PICC inserted into a patient. Processing device 112 is in communication with display device 110. Those skilled in the art can appreciate that display device 110 may include any type of device for presenting visual information such as, for example, a computer monitor or flat-screen display. Display device 110 may be equipped with user input devices, such as buttons for silencing audible alarms, erasing visual alarms or a combination thereof. Processing device 112 may be operably coupled to an ECG or be integrally formed therewithin. Those of skill in the art will also appreciate that display device 110 may be operably coupled to the ECG or may comprise the display device of he ECG.

In one embodiment, sensor 114 may include electrodes for measuring ECG signals microphones for measuring and recording heart sounds, sensors such as sound sensors, electrocardiography sensors, optical finger sensors, optical magnetic sensors and the like. While FIG. 4 depicts one sensor 114, those of skill in the art will appreciate that more than one sensor 114 may be used. Sensor 114 is used to convert electrical signals from the heart to an atrial and waveform having particular waveform attribute of the patient being monitored. The output from the sensor can alternately or simultaneously be an electrical output signal configured to be received by processing device 1 and converted to a physical output, including but not limited to graphical display, printout, and the like. Those skilled in the art can appreciate that the number, type and use of sensors may vary.

The processing device 112 includes non-volatile memory. The processing device computes the position and direction of the catheter tip in real form and converts the electrical signals from sensor 114 into indicia representative of the peak, deflection and transition points of the associated P wave. In one aspect, the indicia may comprise a graphical bar form, as shown, as the catheter tip progresses toward the heart. Those of skill in the art will appreciate that the bar may be vertical or horizontally placed or alternatively the graphical form may comprise a line that runs along an X and Y axis. Still yet alternatively, the gauge may be circular. In whatever representation chosen, the indicia will have a range according to the maximum QRS waveform.

The system and method in accordance with the invention determines whether a P wave is present, whether they are occurring regularly, whether the P wave is normal/standard or non-standard, whether the P waves are smooth, rounded, upright or inverted and whether the P waves have uniform or non-uniform shapes, and P wave intervals, which shorten with increased heart rate, including whether they are constant or vary.

The system includes at least one sensor capable of sensing the electrical signal associated with the P wave. The sensor is configured for attachment on the tip of the peripherally inserted central catheter or in a lumen thereof and configured to provide a waveform signal to a processing device. The waveform signal may comprise a P wave including the initial deflection, the maximum height or peak, a negative deflection and a transition point. The transition point is the point between the negative deflection and the peak and indicates correct positioning of the PICC. The system also includes a database including known data corresponding to standard and non-standard P waves including peaks, deflections and transition points thereof. A processing device is in operable communication with the sensor and the database and is configured to receive the at least one waveform signal and calculate waveform attributes of the waveform including the initial deflection of a P wave, the maximum peak, the secondary deflection and the transition point. The processing device further compares the waveform attributes with the database of known data and output a graphical representation of the maximum peak, the secondary deflection and the transition point. The system in accordance with the invention also includes a display device configured to display the output and may comprise a computer screen, a flat-screen display, a projector, a printing device and/or an audible device. The display device may also have user input device configured to communicate with the display device and the processing device.

The database may also include a plurality of waveforms and waveform attributes such as P waves, QRS complex, PR intervals, PR segments, AT intervals, ST segment and the T wave.

### The Processing

The computing system may be operative to generate indicia representing a real time image of the P wave.

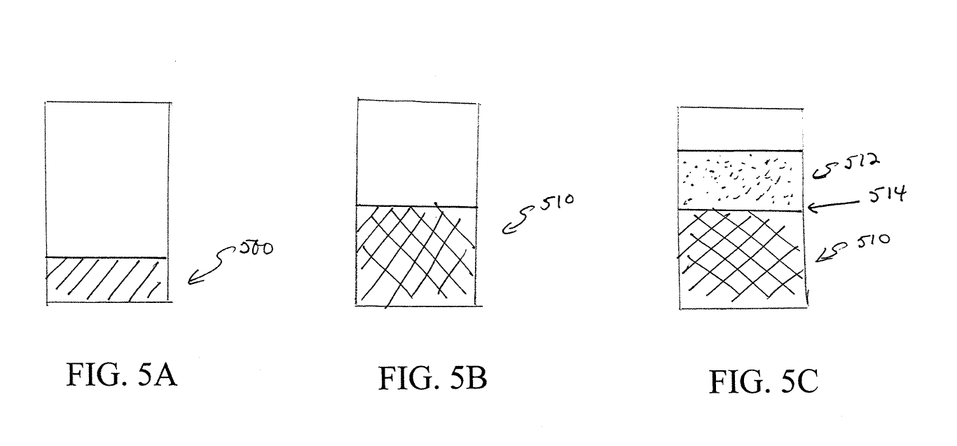

As seen in FIGS. 5A the processing device 112 has converted the monitored P wave of a patient to a graphical representation 500. In FIG. 5B the maximum P wave peak 510 is depicted at the midpoint of the graph. FIG. 5C graphically illustrates the deflection of the P wave 512 as the catheter tip is pulled back from the entrance to the right atrium of the heart. The transition point 514 is graphically displayed and can be easily seen by a user.

The bar graph may be colored and progress from zero and increase as the P wave increases towards the right atrium. When the processing device 112 detects the negative deflection of the P wave the color of the bar may change indicating to a user that the deflection is increasing. The colors may be red and green but those of skill in the art will appreciate that any color combination may be used. The catheter tip can then be retracted to the maximum P wave position and the ideal anatomic infusion location of the patient.

It will be appreciated by those skilled in the art that changes could be made to the embodiment described above without departing from the broad inventive concept thereof. It is understood, therefore, that this invention is not limited to the particular embodiment disclosed, but it is intended to cover modifications within the spirit and scope of the present invention as defined by the appended claims.

What is claimed is:

1. A system for guiding the placement of a peripherally inserted central catheter in a patient comprising:
a peripherally inserted central catheter having a tip and a lumen therewithin;
at least one sensor configured to be coupled to said catheter and structured to sense and transmit an electrical signal associated with a P wave;
a data base including known data corresponding to standard and non-standard P waves including peaks, deflections and transition points; and
a processing device in operable communication with said at least one sensor and said data base, said processing device configured to receive said electrical signal and identify P waveform attributes of said signal, cross-reference said waveform attributes and said electrical signal with known data in said base data, and output indicia representative of said P waveform attributes and said signal based on said cross-reference.

2. The system of claim 1 further comprising a display for displaying the indicia representative of said P wave.

3. The system of claim 2 wherein said indicia comprises a bar graph.

4. The system of claim 3 wherein said bar graph represents a maximum peak of a P wave.

5. The system of claim 3 wherein said bar graph represents P wave deflection.

6. The system of claim 3 wherein said bar graph is horizontally placed on the display.

7. The system of claim 3 wherein said bar graph is vertically placed on the display.

8. The system of claim 4 wherein said maximum peak is represented by a color different than a color of the graphical representation of the P wave deflection.

9. The system of claim 2 wherein a transition point between a graphical representation of P wave deflection and a maximum P wave peak is depicted for precise catheter placement.

10. The system of claim 1 wherein said processing device is operably coupled to an electrocardiogram device.

11. The system of claim 2 wherein said display means is operably coupled to an electrocardiogram device.

12. The system of claim 1 wherein said at least one sensor is positioned within the lumen of the catheter.

13. The system of claim 1 wherein said at least one sensor is positioned on the tip of the catheter.

14. A method for guiding the placement of a peripherally inserted central catheter in a patient comprising:
providing a peripherally inserted central catheter having a tip and a lumen therewithin;
coupling at least one sensor to said catheter for sensing and transmitting an electrical signal associated with a P wave;
providing a data base including known data corresponding to standard and non-standard P waves including peaks, deflections and transition points;
providing a processing device for communicating with said at least one sensor and said data base, said processing device for receiving said electrical signal from said at least one sensor and identifying P waveform attributes of said signal, cross-referencing said waveform attributes and said electrical signal with known data in said base data, and outputting indicia representative of said P wave and P waveform attributes based on said cross-reference.
